(19) United States
(12) Patent Application Publication
(10) Pub. No.: US 2008/0091238 A1
(43) Pub. Date: Apr. 17, 2008

(54) Electrochemical Compensation Oscillator For The Biological Protection Of Living Organism

(76) Inventor: Isabel Helene Esabo Colliard, Douvaine (FR)

Correspondence Address: OSTROLENK FABER GERB & SOFFEN, 1180 AVENUE OF THE AMERICAS, NEW YORK, NY, 100368403, US

(21) Appl. No.: 11/574,572

(22) PCT Filed: Jul. 25, 2005
(86) PCT No.: PCT/FR05/01923

§ 371 Date: Aug. 15, 2007

(30) Foreign Application Priority Data
Jul. 23, 2004 (FR) 0408177

(51) Int. Cl. A61N 1/16

(52) U.S. Cl. 607/3

(57) ABSTRACT

The approach consists in:
analyzing a first signal corresponding to electromagnetic radiations emitted by the appliance,
producing a second signal which differs from the first signal by an inversion relating to at least one signal parameter, and
applying the second signal, in electromagnetic radiation form, to the compensation medium.

The inversion can relate to at least one of the following signal parameters:
the phase,
the polarity of the return wave,
   and can be produced by a digital processing of said signal.

The invention also relates to a support containing a quantity of duly treated medium, intended to be joined to an appliance that is a source of polluting radiation, and such an appliance equipped with this support.

The invention relates to protection against the athermal biological effects of the undesirable non-ionizing radiations emitted by electrical and electronic appliances, such as cordless telephones, computers, cathode ray tube image transmission systems, portable telephones, the relays of electronic organizers, radio link-based communicating interfaces, such as Bluetooth or WiFi interfaces, video games, microwave ovens, domestic and industrial electrical appliances, vehicles and other electronic appliances.

The field of the invention moreover extends to all non-ionizing radiating appliances, including radio communications. In this sense, it adds reliability and enhances the techniques for using saline solutions already described, in particular in patent documents WO-A-93/25270, WO-A-02/097915, FR-A-2 825 519 and EP-A-0 646 033.

For information, the techniques used by the above-mentioned patents aim to physically connect to a radiating appliance of the above-mentioned type—so-called “polluting” appliance—a vessel containing a small quantity of compensation medium, which has previously been submitted to a non-ionizing irradiation having roughly the same characteristics as that emitted by the polluting appliance. As an indication, the medium concerned can be a liquid, such as water, containing metallic salts with a maximum concentration of 20 mg of salt per 100 1 of medium. In a typical application, approximately 150 ml of medium is encapsulated in a tube made of glass, or of another chemically and electrically neutral material, itself contained within a non-ferrous metal enclosure, for example aluminum. This enclosure is incorporated in a support element designed to be fixed to a part of the body of the “polluting” appliance, such as a portable telephone terminal, or inside the latter.

A number of techniques are described by these documents for subjecting the medium to the non-ionizing irradiation during its preparation phase. Thus, the above-mentioned document WO-A-93/25270 proposes placing a so-called Lecher antenna above a vessel containing an aqueous medium. This antenna has two branches linked by a resonance loop which can be tuned to a required frequency by means of a mobile strip. One of the branches is held above the vessel by an operator in order to produce a resonant coupling between the latter and the aqueous medium, and to apply a non-ionizing irradiation thereto. This same document proposes as a variant the emission of a coherent radiation emitted by laser, connected to means for cooperating with the hand of the operator. These means can be, for example, a loop formed in the laser power feed line, and through which the operator passes his hand in such a way as to provide, here too, a coupling between his hand and the aqueous medium, which is in this case illuminated by the laser. This technique, involving the hand of the operator in the kinematic chain, is problematic, because it does not involve any system to make it possible to control its repetitiveness and reliability, and to establish the countermeasure according to the double-blind principle.

Similarly, the above-mentioned document WO-A-02/097915 proposes irradiating the medium by placing it in a vessel above which is placed a radiation-emitting appliance, the characteristics of which are similar to those of the radiation against which protection is required. The polluting appliance can be a mobile telephone terminal in its normal operating state, so as to roughly reproduce all the radiations of the appliance for which the medium is intended to provide the protection. This operation is then complemented by a polarized irradiation, produced by an oscillator, via a monopole-type antenna, also placed above the vessel. The output frequency of the oscillator is an exact copy of the basic frequency emitted by the above-mentioned appliance, with an intensity of a few microvolts per meter. The signal from the oscillator passes through a magnetized rod installed on the antenna circuit.

While these procedures produce technical effects that are convincing and have been experimentally verified, there does, however, remain a vital need to redefine them even more scientifically in order to increase their effectiveness and their reproducibility, and to obtain a finer control of the activation parameters of the medium.

The present invention proposes a novel approach according to which the electromagnetic compensation system or oscillator is considered as an “animat” or a “programmed animal-robot” immersed in an environment and using, for all the control phases, the stimuli received from this environment and reacting by different processes. The latter can comprise: absorption, anechoic effect, anelectrical effect, physical-chemical excitation, etc. The system can thus interact, in the artificial intelligence sense, with its aggressor (polluting electromagnetic system) so as to significantly reduce the polluting effects, or even eliminate them.

Moreover, the present approach makes it possible to respect the principle of co-dependence. In practice, the system or oscillator behaves like an animat confronted with a continuous stream of stimuli immediately the polluter is activated. To “survive”, it needs to be able to extract the relevant criteria from this stream and deduce from them the most appropriate behavior. To do this, the proposed system is neuro-anatomically or ethologically inspired, enabling it to memorize the reactivity of the living organism (man, animal, etc.) by a reactive control which relies on a set of simple sensory-motive loops, associated with identified basic behaviors. This type of behavior can be summarized by a compensation system or oscillator immersed in the environment close to the polluter and using, for all the control phases, the stimuli received from that environment, then by its incorporated functions, resources for countering the polluter. This countering action can be obtained:
by an action-reaction procedure, as in the case of the physical-chemical solution provided with a memory enabling itself to become an emitter in phase opposition to the polluter (saline solution comprising earthy alkaline salts and ferromagnetics doped by catalytic action by a nascent oxidant) and/or
by a so-called double-blind procedure (physical-chemical detection (with or without photonic crystals) and electro-computer detection) making it possible to synthesize the process to be retained to reduce or eliminate the harmful electromagnetic waves from the polluter.

As an example, the electromagnetic compensation oscillator or system can comprise a volume of liquid “saline solution”, gelled or solid in colloidal or microcrystalline suspension, charged by a specific electromagnetic transfer treatment and doped according to the required protection with ferromagnetic photonic crystals and/or the presence of a catalyst comprising a nascent oxidant (for example, nascent oxygen) in order to ensure on the one hand a longevity and active stability of the solution, and on the other hand an exchange if necessary with the part of the system responsible for controlling the double-blind function, then, if necessary, fully replacing the presence of the saline solution.

The saline solution can be captive in a vessel made of non-ferromagnetic material that is chemically neutral and suited in its volume configuration with all the electronic or digital part (design) to the radiating “polluting” appliance to be “neutralized” regarding its external radiation, to placement on or near the latter, even to incorporating it in the polluting appliance, in accordance moreover with the electromagnetic compatibility directives regarding other nearby electrical or electronic appliances.

As an indication, the initial electromagnetic charge can be done:
by transferring the radiation from the calibration appliance to the saline solution, via the connection through a “balloon” of two electrically conducting coils or cylinders, respectively enclosing the polluting-type appliance and the saline solution container. (The term “balloon” denotes a Mobius antenna-loop connection process) in order to emit spatial electromagnetic field radiations.) The “balloons” are interlinked by a conductive cable capable of transmitting the frequencies required for the fields being treated; and/or
by an electronic and computerized process controlling a well known generator of electromagnetic fields in the range of frequencies for which protection is required.

In order to ensure the good operation of the assembly and control of its repetitiveness, the transfer system can be complemented with an amplifier and a phase inverter between the coil of the “polluter” and the “doped saline solution” coil in this version, or with control by control of the protection oscillator in an all-digital or hybrid version.

The invention can make it possible to put in place the double-measurement and/or the double-activation principle, the one being perfectly controlled by minimizing, even cancelling, the operator effect.

In a possible embodiment of the invention, it is possible to obtain a computerized control. Moreover, it is possible to introduce catalysts by fixing saline solution (active or so-called “nascent” oxygen) to confer an additional protection in the system of biocompatibility used for biological protection or feedback (wave absorber) of the polluting system.

It is possible to envisage a high quality control, because it is electronic, of the saline solutions. It is possible to obtain an excellent stabilization in their life by injecting or introducing O- into the saline solution (permanent electromagnetic effect known in the soluble zone).

Moreover, in the preferred embodiments, there is no manipulation likely to add an interfering radiation specific to the manipulator.

The magnetic irradiation of the saline solution is established automatically by the electronic recognition of the polluting interference frequencies.

More particularly, according to a first object, the invention proposes a method of preparing a compensation medium intended for protection against the biological effects of the electromagnetic radiations emitted by an appliance, characterized in that it comprises the steps of:
analyzing a first signal corresponding to electromagnetic radiations emitted by the appliance,
producing a second signal which differs from the first signal by an inversion relating to at least one signal parameter, and
applying the second signal, in electromagnetic radiation form, to the compensation medium.

When the compensation medium is used subsequently with a “polluting” appliance, for example in a support incorporated in the latter, it can absorb—or have absorbed —the polluting signal from said appliance by the production of an absorption spectrum within, or in the immediate environment of, this appliance. Naturally, it will be necessary to ensure that, if necessary, this spectrum remains compatible with the natural working frequency of the appliance (for example a mobile telephone) in order for it to be able to fulfill its function.

The inversion can relate to at least one of the following signal parameters:
the phase,
the polarity of the return wave,
at least a part of the spectrum.

Inversion, in particular cases, can be replaced by a polluting wave absorption spectrum generation, by means of an appropriate generator of polluting wave absorption spectra.

In an embodiment, the inversion is produced by a digital processing of the signal.

The inversion can comprise at least one out of:
a Fourier transform,
a Merlin-Fourier transform,
a Laplace transform, in particular for the relatively high frequencies.

The second signal can be produced by transforming the first signal by means of an electronic processing device, or by synthesis.

In the latter case, the second signal can be produced from parameter control data for a reference generator operating in the frequency range for which protection is required.

The method can comprise the steps of:
extracting the first signal from the electromagnetic emission from a source appliance roughly of the same type as the appliance for which the protection is intended, the source appliance and the compensation medium being placed respectively in a radiation detecting device and in a radiation emitting device that are mutually matched, the emitting device supplying the signal to the compensation medium from the electromagnetic radiation of the source device,
subjecting the first signal to the inversion upstream of the emitting device, thus transforming it into said second signal,
supplying said second signal as input to the emitting device such that the latter produces in response an electromagnetic radiation with corresponding inversion, received by the compensation medium.

In one embodiment, the relatively high and relatively low frequency bands of the first signal are processed separately.

The radiation in a relatively high frequency band of the second signal can be transmitted to the compensation medium by means of a multi-dipole antenna, and the radiation in a relatively low frequency band of the second signal can be transmitted to the compensation medium by means of a winding.

Preferably, the first signal is also subjected to a filtering for the purpose of eliminating the high-order harmonics (for example, if the polluting appliance permits, using an absorption spectrum surrounding the polluting system).

In one embodiment, the compensation medium is also subjected to a treatment by a nascent oxidant, for example nascent oxygen.

It is then possible also to provide for a step for defining the stability of the compensation medium, by analyzing the nascent oxygen in the latter.

According to a second aspect, the invention provides for a method of protection against the biological effects of the non-ionizing electromagnetic radiations emitted by an appliance, characterized in that it comprises the steps of:
preparing a compensation medium according to the first aspect, where said first signal corresponds to the electromagnetic radiation emitted by the electromagnetic radiation appliance,
packaging a quantity of the duly prepared compensation medium in a support, and
physically connecting said support to said appliance.

According to a third aspect, the invention provides for a device specifically provided for the preparation of a compensation medium intended for protection against the biological effects of the non-ionizing electromagnetic radiations emitted by an appliance, characterized in that it comprises:
means for analyzing a first signal from the electromagnetic radiations emitted by the appliance,
means for producing a second signal which differs from the first signal by an inversion relating to at least one signal parameter, and
means for applying the second signal, in electromagnetic radiation form, to the compensation medium.

The optional characteristics presented in the context of the first or the second aspect of the invention can be applied mutatis mutandis to the appliance according to the third aspect.

According to a fourth aspect, the invention relates to a support intended to be physically connected to an appliance emitting non-ionizing electromagnetic radiations, characterized in that it contains a quantity of compensation medium treated in accordance with the method according to the first aspect.

According to a fifth aspect, the invention relates to an appliance emitting non-ionizing electromagnetic radiations, characterized in that it incorporates the support according to the fourth aspect.

The preferred embodiment uses a compensation oscillator that is matched in frequency, impedance and phase to certain polluting appliances, and extends the technological developments and applications to all appliances recognized as electromagnetic biological polluters, by the principle of a compensation channel associated with a procedure or a systematic control channel.

The invention and the advantages it provides will become more clearly apparent from reading the description that follows of the preferred embodiments, given purely by way of non limiting examples with reference to the appended figures, in which:

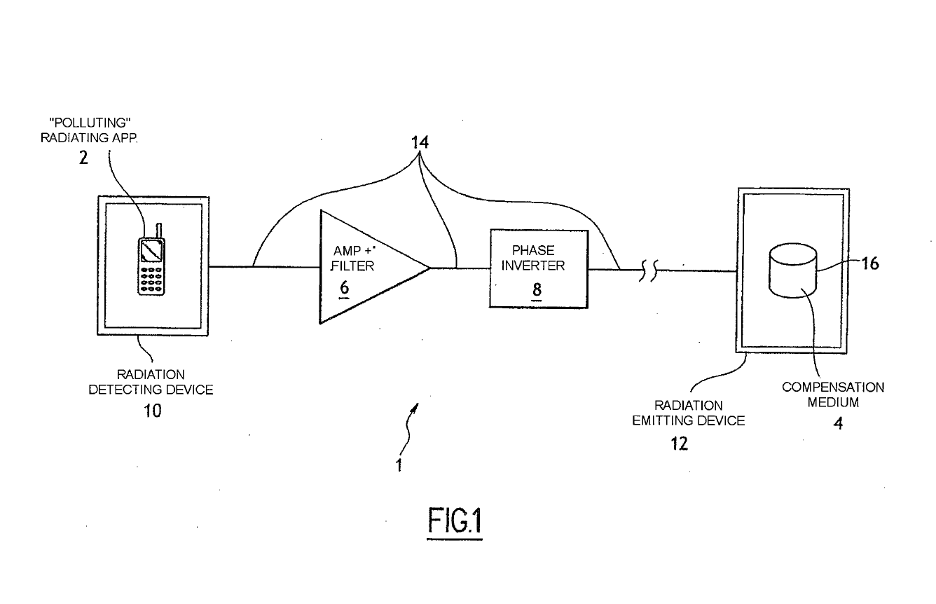

FIG. 1 is a theoretical block diagram showing the basic elements involved in the preferred embodiment,

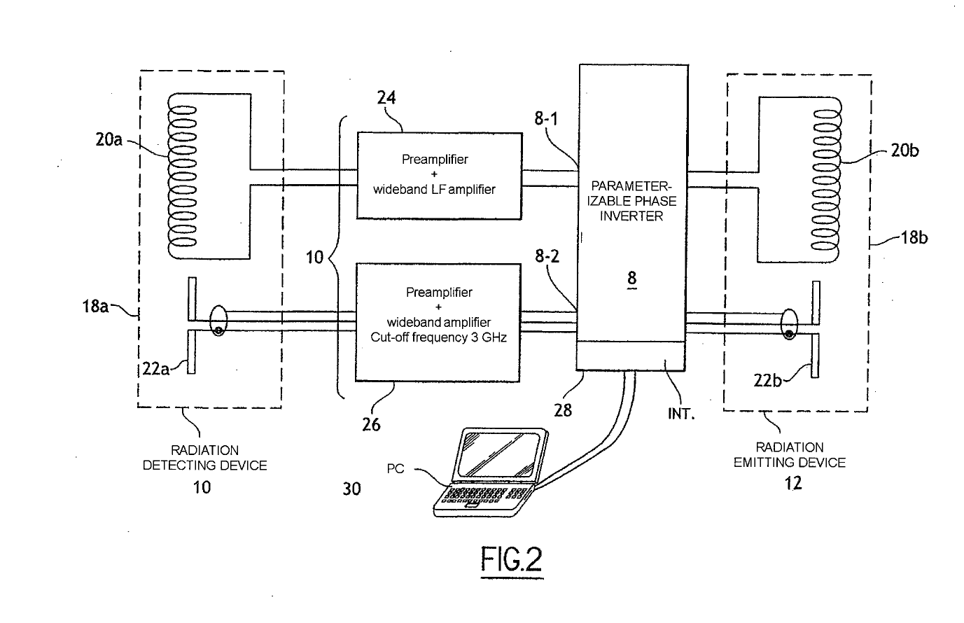

FIG. 2 is a more detailed theoretical block diagram of the embodiment of the invention, showing in particular two amplification, inversion and filtering channels, respectively for the radiofrequencies and the low frequencies,

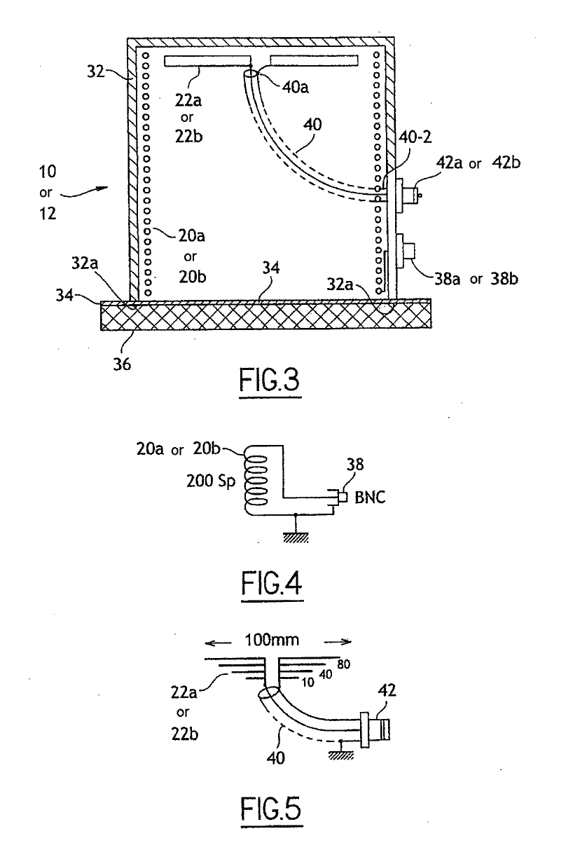

FIG. 3 is a cross-sectional view of a radiation detecting device or a radiation emitting device used in the embodiment of the invention,

FIG. 4 is an electrical equivalence diagram of the low-frequency part of the device of FIG. 3,

FIG. 5 is an electrical diagram of the multi-dipole antenna and its electrical connection forming the radiofrequency part of the device of FIG. 3,

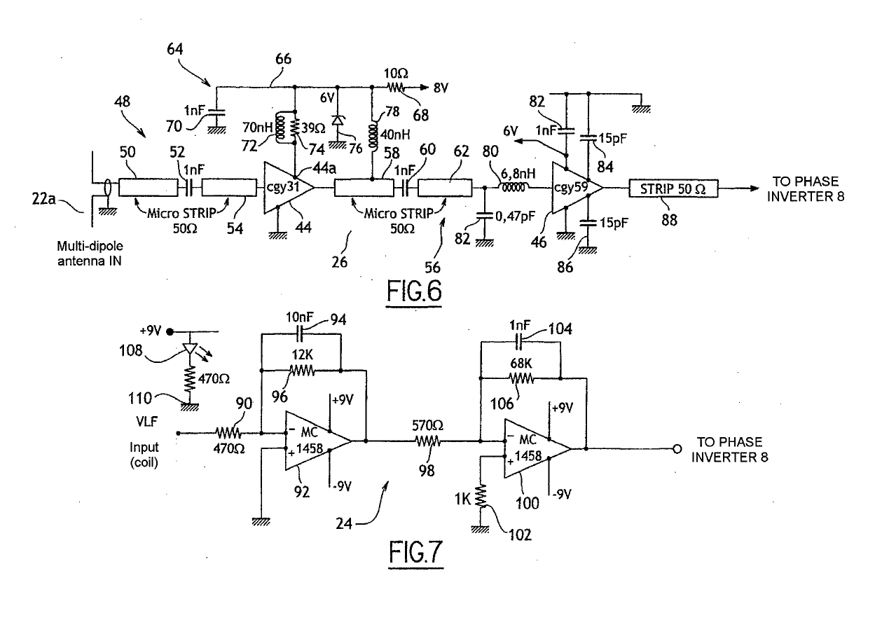

FIG. 6 is an electrical diagram of the amplification and microwave filtering channel of the embodiment, with connection to a digital phase inverter,

FIG. 7 is an electrical diagram of the amplification and low-frequency filtering channel of the embodiment, with connection to a digital phase inverter, and

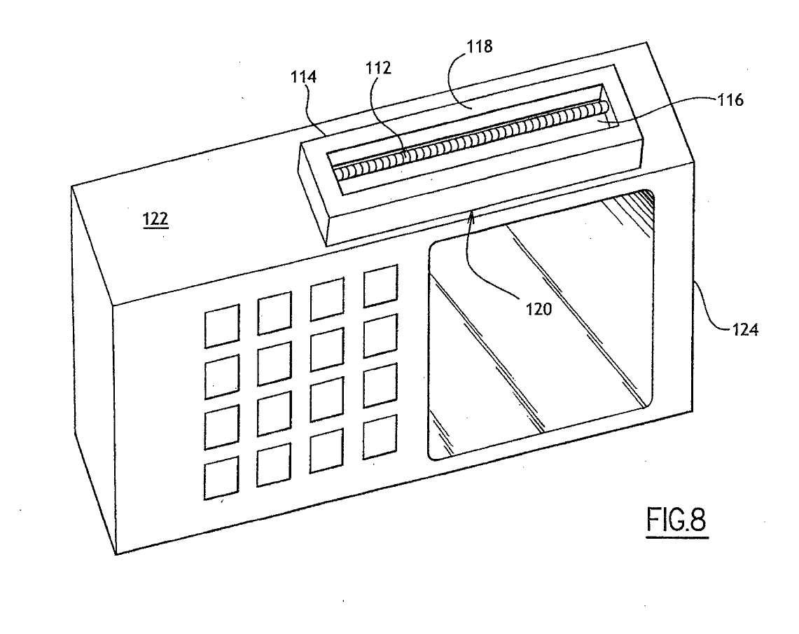

FIG. 8 shows a support containing a vessel of treated compensation medium, and the electronic command and control system systematically activated by the electromagnetic waves from the polluters according to the invention, in this case a mobile telephone terminal, the assembly being joined to an appliance emitting polluting electromagnetic radiation.

As shown by FIG. 1, the embodiment provides for a system 1 by which a so-called “polluting” radiating appliance 2, in this case a mobile telephone terminal, is electrically coupled over its radiating frequencies to a compensation medium 4, with the successive placement of an amplification and filtering 6 and inversion 8 assembly. These inversion means produce an inversion, total or partial, on at least one of the parameters of the signal produced by the polluting radiation, for example its phase or its spectrum, as in the case of the embodiment.

The polluting appliance 2 and the compensation medium 4 are placed respectively in a radiation detecting device 10 and a radiation emitting device 12, separated by a length of double-braided coaxial interconnecting electric cable 14, to which are connected the amplification and filtering 6 and inverter 8 assembly.

The amplification and filtering assembly 6 receives the signals from the polluting appliance 2 at the output of the detecting device 10 on two independent channels, respectively dedicated to the radiofrequency spectrum and to the low-frequency spectrum (FIG. 2). The inverter 8, here shown between the emitting device 12 and the amplification and filtering assembly 6, performs, in a manner known per se, a Fourier or Merlin-Fourier transform, or even a Laplace transform (the latter in particular for the relatively high frequencies), to produce a spectral inversion of the signal before driving the emitting device 12.

The principle defined by the term “inverter” is in this case an assimilation to a propagation in a monopole or semi-infinite system in which the wave, on arriving at the end in the “air”, is reflected by change of sign and therefore is inverted (often according to the terminal impedances by doubling its intensity) without necessarily changing the phase speed. The term “inverter” is used to mean: inversion of the polarity of the return wave being composed on the next incoming wave, creating, in the Varignon polygon sense, a very much lower resultant (principle of the application of a virtual plug circuit), or a phase opposition. However, this can also be understood to be an anechoic effect with a small part of the wave continuing to be propagated, particularly in an approach according to which the inversion is applied, according to a variant, by a signal synthesis, which can be produced by electronic and computerized means, for example using artificial intelligence techniques.

The compensation method thus allows for a transfer of the waves emitted by the polluter compensated in phase opposition.

The compensation medium 4 is thus activated with the inverted form of the signal emitted originally by the polluting appliance 2, with inversion created by an external electronic processing of the signal.

In the embodiment, the compensation medium 4 is a saline solution, possibly enriched with chemical catalysts. This solution is placed in a vessel 16 within the radiation emitting device 12. As an example, the solution can be a liquid, such as pure water, to which is added a metallic salt, or a combination of metallic and earthy alkaline salts, diluted to a maximum concentration of 20 mg of salt(s) per hundred liters of liquid. This solution is then doped by a powerful oxidant introduced in nascent form, for example nascent oxygen. For the salt, or for one of the salts, it is possible to use an alkaline metal or earthy alkaline salt, and therefore the effective interaction section). The salts of the saline solutions used can advantageously be chlorides of potassium, magnesium or sodium.

Thus, with the system 1, the electromagnetic treatment of the saline solution is done on transferring the radiation from the polluting appliance 2 in operation to the vessel 16 containing this saline solution 4, and after phase inversion of the whole of the spectrum of the polluter.

Advantageously, the cable link 14 maintains the radiation detecting 10 and emitting 12 devices sufficiently far apart for there to be no electromagnetic interference between them.

The time needed for an effective transfer from the polluter 2 to the solution 4 depends on the electrochemical composition of the latter, and can take several minutes or tens of minutes.

A verification of the transfer involves measuring the resistivity of the aqueous solution. It is not, however, necessary in routine operation.

If using a liquid saline solution, it can be subjected to a succussion process described in the patent documents cited in the introductory part, in order to “fix” in time the new electromagnetic characteristics of this solution. Of course, this possibility does not apply for the case where a non-liquid compensation medium, particularly in gelled or solid form, is used.

The signal processing subsystem of the system of the preferred embodiment is represented in more detail in FIG. 2.

The radiation detecting 10 and emitting 12 devices are roughly identical; in the figures that follow, their common elements are identified by the same reference numbers, followed by the suffix “a” for the detecting device and the suffix “b” for the emitting device.

Each detecting 10 and emitting 12 device comprises an enclosure 18a, 18b made of a metallic structure which forms a Faraday cage. Inside the enclosure there are placed:
an electric winding 20a, 20b, for example of the “balloon” type, intended to interact with the part of the radiation in the low-frequency spectrum (up to a few hundreds of kHz) and
a multi-dipole antenna 22a, 22b intended to interact with the part of the radiation in the radiofrequency spectrum, typically up to a few GHz.

The signals relating to the winding and to the antenna are processed on separate amplification and filtering channels, respectively denoted 24 and 26, each comprising a preamplifier and a wideband amplifier. These two channels 24, 26 together fulfill the function of the amplification and filtering block 6 in the diagram of FIG. 1. In the example, the radiofrequency amplification and filtering channel 26 has a cut-off frequency located at 3 GHz. However, it is possible to envisage higher processing frequencies, depending on the applications, for example reaching 40 GHz.

The output of each of the two amplification and filtering channels 24, 26 is supplied to an input of a respective inversion channel 8-1 and 8-2 of the phase/spectrum inverter 8, to be subjected to a phase/spectrum inversion before driving the radiation emitting device 12. This phase/spectrum inverter 8 is of the parameterizable type, produced using digital technology. To this end, it has an interface 28 with a port for connecting to a PC-type computer 30, the latter being equipped with software dedicated to controlling the inverter, making it possible to establish the various inversion parameters. Advantageously, the inversion produced in this way is a Fourier transform or a Merlin-Fourier transform, or even a Laplace transform.

The physical implementation of a radiation detecting 10 or emitting 12 device is represented in FIG. 3.

The device 10 or 12 comprises an external conductive chamber 32, cylindrical in shape, which provides the Faraday cage function, for insulating the interior from electromagnetic interference. The conductive material of the chamber is advantageously mu-metal, this metal also offering a very strong internal insulation against the external magnetic fields.

The chamber 32 rests freely by its lower edge 32a on a copper plate 34, which provides the bottom conductive plane of the Faraday cage. This plate 34 is placed on a relatively thick insulating substrate 36, made of ebonite, for example.

In the example illustrated, the electrical winding 20a or 20b follows the internal wall of the chamber 32 over roughly all its height. It comprises approximately 200 turns of 6/10 wire wound on 200 mm diameter PVC to be, making it possible to tune to the low frequencies that can drop below the ELF (Extra Low Frequency) spectrum. It is connected by one of its ends to the conductive core of a BNC-type electrical connector 38, suited to the ELF frequencies, the other end of the winding being linked to the body of the chamber 32, which constitutes a ground plane. This connector 38 is physically mounted on the wall of the chamber 32 in order to allow a connection to the winding from the outside.

As a variant, the winding can be of the “balloon” type, known per se in the oscillating signal field. For information, a “balloon” winding is a magnetization field H transformer. The winding is then produced using two coaxial cables that are crossed and define a spheroid. More particularly, a first cable runs through a half-circle of the section of the spheroid and is connected to the second cable which runs through the complementary half-circle. The connection links the core of the first coaxial cable with the enveloping braid of the second, and vice versa, giving rise to diametrically opposed crossing points. These crossings are overlaid, firstly on the diameters increasing from the bottom pole to the equatorial plane of the spheroid, then decreasing to reach the top pole.

The antenna 22a or 22b is of the multi-dipole type, tuned to the frequencies in the 100 MHz to 3 GHz spectrum. It is centered in the axis of the chamber 32 and fixed near to its top part. The opposite connections of the antenna are respectively linked to the conductive core and to the enveloping conductive braid of a coaxial cable 40, at a first end 40-1 of the latter. The other end 40-2 of this cable is linked to an N-type connector 42 for radiofrequencies, physically mounted on the wall of the chamber 32, just above the connector 38, in order to allow a connection to the antenna from the outside. The connectors 38 and 42 thus constitute interfaces between the electromagnetic radiation and the signal that corresponds to this radiation, this signal being conducted by a cable 14 (see FIG. 1). In the embodiment, these connectors each have an impedance of 50 ohms.

FIG. 4 represents the electrical equivalence diagram of the winding 20a or 20b and its connection to ground and to the BNC connector 38 as described above.

FIG. 5 represents the electrical equivalence diagram of the antenna 22a or 22b and its connection to ground and to the connector 42, as described above. In the example, the antenna comprises four dipoles, stacked and symmetrical, respectively of 10 mm, 40 mm, 80 mm and 100 mm radius (working from the bottom dipole to the top dipole). For each dipole, one branch is linked to ground via the braided conductor of the coaxial cable 40, the other being linked to the central conductive core of the latter.

All the cables carrying the signals from or to the antennas 22a, 22b and the windings 20a, 20b, are preferably of the double-braided coaxial type. This concerns the cables 40 within the chambers 32 of the radiation detecting 10 and emitting 12 devices, and link cables 14 between the chambers 32 and the blocks 24, 26, 8 of FIG. 2. As a variant, the latter can be produced in waveguide form.

The electrical diagrams of the amplification and filtering channels 24 and 26 are respectively represented in FIG. 6 and in FIG. 7. In these figures, the values indicated for the active electronic components are preferably within tolerances less than 5%. The values of the passive components (capacitors, resistors, etc.) preferably have a tolerance less than 1%.

The radiofrequency signal amplification and filtering channel 26, represented in FIG. 6, comprises a preamplifier 44 followed by an amplifier 46. The input of the preamplifier 44 is linked to the output of the multi-dipole antenna 22a, on the connector 42a (FIG. 3), via an impedance matching device 48, which in particular ensures that the amplification occurs without the wave being distorted. This device 48 comprises a series assembly comprising, successively from the antenna output: a first microstrip element 50, a capacitor 52 and a second microstrip 54. The latter is directly linked to the signal input of the preamplifier 44, which is a radiofrequency integrated circuit in AsGa technology, such as the model known by the reference CGY31.

The preamplifier output 44 is transmitted to a second impedance matching device 56, successively comprising a first microstrip 58 which receives the output signal, a capacitor 60 and a second microstrip 62, in a series assembly identical to the first device 48.

The preamplifier 44 also comprises a frequency-filtering input 44a, to which a filtering LCR device 64 is linked. The latter comprises a voltage power supply line 66 linked to a +8V source via a resistor 68, and to which are connected:
one plate of a capacitor 70, the other plate of which is grounded,
the first common parallel-connection node of an inductor 72 and a resistor 74, the second node being linked to the input 44a of the preamplifier 44,
the anode of a zener diode 76, the cathode of which is grounded, and
a first terminal of an inductor 78, the second terminal of which is linked to the center of the first microstrip 58 of the second matching device 56.

The cut-off frequency of the filtering LCR device 64 is fixed by the specific values of the components; those indicated in the diagram correspond to a cut-off frequency located in the region of 3 GHz. The circuit provides in particular for a frequency chopping, with a portion of the harmonics above order 3 or 4 eliminated, in order to retain only the first-order harmonics.

The zener diode 76 is used to prevent return phenomena between the two filtering stages of the device 64 (the other being associated with the amplifier 46), and in particular those due to a frequency differential.

The output of the second matching device 56, taken from the second microstrip 62, is connected to the signal input of the amplifier 46 via an inductor 80. The terminal of the inductor that is linked to the microstrip 62 is also connected to a first plate of a low-value capacitor, the other plate of which is grounded.

As for the preamplifier 44, the amplifier 46 is in AsGa technology, in this case, the model known by the reference CGY59. The amplifier 46 makes it possible to supply as output the portion of the frequency and its low-order harmonics with sufficient intensity for the processing.

At this amplification stage, the signal is once again filtered and cleaned by means of capacitors 82, 84, 86 linked between the filtering control inputs of the amplifier 46 and ground.

The output of the amplifier 46 is presented, via a 50 ohm microstrip 88, to the input of the radiofrequency channel 8-2 of the inverter 8. The output of this channel reproduces the inversion of the amplified signal on the radiofrequency connector 42b of the radiation emitting device 12. This way, the antenna 22b of the latter is driven with the same radiation as that received by the antenna 22a of the detecting device 10 from the polluting device 2, but in a filtered form, cleaned of the high-order harmonics, amplified and inverted in phase/spectrum. By receiving this signal from the antenna 22b, the compensation medium 4 thus becomes activated by an electromagnetic radiation originating from the polluting device 2. Because of this, when this medium is used to compensate the damaging effects of the radiation from a particular type of polluting device (in radiation characteristics terms), the compensation will be matched.

The amplification channel 24 for the low-frequency (ELF) signals, represented in FIG. 7, is of a design similar to that of the amplification channel 26 for the radiofrequency signals.

The output of the BNC connector 38a linked to the winding 20a is presented, via a resistor 90, to the inverting (negative) input of a first operational amplifier 92, the non-inverting (positive) input of which is linked to ground. This operational amplifier 92, in this case the model known by the reference MC 1458, operates as a preamplifier with active filtering. To this end, its output is looped to the inverting input via a parallel arrangement of a capacitor 94 and a resistor 96. The output of the first operational amplifier 92 is presented, via a resistor 98, to the inverting input of a second operational amplifier 100, identical to the first, and the non-inverting input of which is linked to ground via a resistor 102. This second operational amplifier 100 constitutes an amplification stage with filtering, having its output looped to the inverting input via a second parallel arrangement of a capacitor 104 and a resistor 106. The output of the second operational amplifier 100 is directly linked to the input of the low-frequency inversion channel 8-1 of the inverter 8.

This inversion channel 8-1 works roughly in the same way as the radiofrequency inversion channel, and produces as output the inversion of the amplified signal on the low-frequency connector 38b of the radiation emitting device 12. In this way, the winding 20b of the latter is driven with the same radiation as that received by the winding 20a of the detecting device 10 from the polluting device 2, but in filtered form, cleaned of the high-order harmonics, amplified and inverted. By receiving this signal from the winding 20b, the compensation medium 4 thus becomes activated by an electromagnetic radiation, in this case, in the low-frequency spectrum, originating from the polluting device 2. Because of this, when this medium is used to compensate the damaging effects of the radiation from a particular type of polluting device (in radiation characteristics terms), the compensation will be matched.

In order to cover all the emissions from the polluting device 2, the latter is advantageously operated in all its possible different modes. For example, in the case of a mobile telephone terminal, the latter will be put into operation over all the frequency bands that it can cover, set to conversation mode, standby mode, cell hunting mode, TDMA (Time-Division Multiple Access) communication mode, internal switching mode for managing the various tasks, etc.). During these operating modes, the system 1 will be active to transmit the inverted radiation spectrum to the compensation medium 4 by the means described.

The low-frequency processing channel can be used in particular to cover the low switching frequencies of certain electronic appliances, such as mobile telephone terminals using TDMA signals, and other forms of signal management.

Preferably, the frequencies processed by the low-frequency amplification channel 24 can encompass the range from 10 Hz to 250 MHz, so as to correspond to the Earth's magnetic field. In practice, the intensity of the Earth's magnetic field is of the order of 2.5 Tesla, and the impedance of the terrestrial ground is 599 Ohms on average. Thus, plotting a curve of the magnetic field and of the electrical field on the Y axis and the frequency on the X axis, gives the magnetic field which begins at a few Hz and tends towards zero at a frequency of 2 or 3 MHz, and the electromagnetic field which becomes high as from 200 kHz, and which rises with frequency. The magnetic field and electrical field trend curves cross at approximately 1 MHz, this crossing point corresponding to the terrestrial impedance of 599 Ohms.

It can be estimated that the inversion frequency of the electrical field relative to the magnetic field is of the order of 25 to 30 MHz at sensitive points of the human body (brain), located at approximately 1.5-2.0 m above the ground. It is advantageous to process the signal at the frequencies both below and above this frequency, in particular in order to take account of the present altitude, hence the choice of a frequency coverage up to 3 GHz in the embodiment, preferably from the low frequencies as indicated above. The filtering used in the embodiment aims to eliminate the frequencies below 100 MHz, and in particular the extra low frequencies.

The circuit also comprises a light-emitting diode 108 which indicates the presence of voltage. The anode of the diode is linked to the output of the two coils 22a and 22b, whereas its cathode is linked to ground via a resistor 110. This arrangement also makes it possible to indicate, by an off state of the diode 108, the existence of an instability between the electronic channel and the channel comprising the compensation medium 4.

For the two amplification and filtering channels 24, 26, the fact of providing two amplification stages (preamplifier and amplifier) makes it possible to obtain a double filtering, and, in this way, have a tolerance to the characteristic variations of the components between the respective stages. It should be noted that the two filtering functions are operated with slightly different cut-off zones, in order to avoid a low-frequency interference phenomenon.

The inverter 8 works by digital computation using software run by a microprocessor, which performs a Fourier transform and/or a Merlin-Fourier inversion on the signal of each of the radiofrequency 8-2 and low-frequency 8-1 channels so as to obtain the spectrum of these frequencies, or even a Laplace transform, in particular for the low frequencies.

In the example, the inversion algorithm implemented by the inverter 8, in conjunction with the computer 30, performs both a Fourier transform and a Merlin-Fourier transform for the relatively low frequencies, and a Laplace transform for the relatively high frequencies.

To allow for the digital processing, the input of each of the inversion channels 8-1 and 8-2 comprises an analog-digital conversion with sampling frequency suited to the frequency spectrum concerned. The digitized signals are processed in binary data form. The processing can be performed either in real time (as and when necessary) if the computation power is sufficient, or offline. In the latter case, the binary data is stored according to its temporal sequencing to allow for offline processing. After the digital processing to extract the phase inversion, the data passes through a digital-analog converter of the inverter 8, this converter producing the analog drive signals for the emitting device 12. If necessary, it is possible to add an additional amplification stage to the analog output of the inverter 8 to increase the intensity of the drive signal to the emitting device 12 and/or to perform an impedance matching function.

The digital inversion algorithm can be performed in task-sharing mode between the inverter 8 itself and resources within the computer 30. In one implementation, the inverter can be embodied by a computer card comprising the input and output interfaces for each of the two inversion channels 8-1 and 8-2, and in particular the analog-digital and digital-analog converters respectively as input and output. This card can also comprise a processor or a coprocessor cooperating with the processor of the computer, and memories storing the code of the algorithms, parameters programmed by the user and protocols for interfacing with the computer.

As a variant, it is possible to place the inverter 8 upstream of the amplification and filtering channels 24, 26, or between the preamplifier 44, 92 and the amplifier 46, 100 of these channels.

The fact of producing a phase/spectrum inversion by electronically processing the signal is a noteworthy aspect. It is distinguished in particular from the approaches described in the patent documents cited in the introductory part, where the inversion took place in a micro-physical-chemical process in the compensation medium. What is more, the inversion by digital signal processing, in accordance with the embodiment, is particularly advantageous, because it provides for, on the one hand, a great control of the parameters of the inversion algorithm and, on the other hand, a computerized management of the conditions of use. It is thus possible to provide a computerized monitoring of the treated compensation media, making it possible to monitor the trend of their characteristics, etc.

The time during which the radiation from the polluting device 2 is applied to the compensation medium by the system 1 that has just been described, and that is variable according, among other things, to the intensity of the signals applied to the receiving device 12, the frequency bands covered and the quantity of medium to be treated. Typically, the duration is a few minutes or a few tens of minutes, with a quantity of approximately one liter of compensation medium 4 in the vessel 16.

When the compensation medium 4 has been treated in this way by the inverted electromagnetic radiation, it can then be subjected to a succussion step, the object of which is to fix in time the new electromagnetic characteristics of the compensation medium.

Then, the compensation medium 4 is packaged in vessels destined to be added to polluting devices of the same type as that by which this medium has been treated, for example, mobile terminals for the case illustrated.

In the example of FIG. 8 (not to scale), the compensation medium vessel is a tube of aluminum 112, approximately 2 mm in section and 25 mm long, held by its ends in suited recesses in a body made of plastic material 114. If necessary, the medium can be contained in an enclosure of glass or other neutral material, which can in turn be contained in the aluminum tube 112. Preferably, the vessel formed by the tube 112 is made of nonferromagnetic and chemically neutral material, suited to the polluting appliance with which it will be associated, without risk as to its physical-chemical operation.

The tube 112 is placed in a reinforced part 116 on an external side 118 of the body 114. The opposite side 120 of the body is roughly flat and comprises a self-adhesive coating (not shown), by which the assembly can be fixed onto a flat surface 122 of the polluting device 124, the radiations from which are to be compensated (a mobile telephone terminal in the example illustrated). The vessel can also be housed inside the polluting appliance. The tube can be filled by various means, for example by injecting the compensation medium into the tube and crimping both ends before cutting.

In the context of a study on the trend over time of the compensation media, it is proposed to retain in parallel the physical-chemical inversion technique according to the patent documents previously cited, in order to have a counterbalance, or a counterweight, with respect to the novel technique of inversion by signal processing. The comparison between the compensation media for which the inversion results respectively from a purely micro-physical-chemical process (prior art) and from a processing of the electronic signal with inversion according to the invention, makes it possible in particular to refine the parameterizing of the signal processing: initially to match the already proven performance levels of the compensation media according to the prior art, and then to exceed them or adapt them to specific requirements.

The monitoring protocol moreover makes it possible to analyze the factors likely to affect the storage of the radiation by the medium: temperature, time, surrounding radiation, etc.

It is known in particular that the aqueous channel with purely micro-physical-chemical inversion (prior art) maintains the characteristics for a known time, during which it can be used as a calibration basis for the processing channel based on electronic inversion of the radiation signal according to the invention.

The system 1 can be tuned according to the diode 108. To keep the diode lit, the user can make adjustments to the electromagnetic radiation components 18a, 18, 20a, 20b of the radiation detecting 10 and/or emitting 12 devices, for example by increasing or reducing the distance between the antennas.

In one embodiment, the compensation medium 4, in this case in solution form, is preprocessed by making it pass through nascent oxygen. This way, the medium becomes charged with negative ions, the latter giving it a far greater capacity to absorb the electromagnetic charge of the polluting field. A greater stability is also obtained.

A nascent oxidant, such as oxygen obtained from a reaction on oxylite, for example, increases if it is placed rapidly in the liquid medium, at the instant when the example will be made hermetically closed, therefore rapidly under light pressure. There is then obtained a significant increase in free electrons, for which the effective absorption section of the electromagnetic radiation induces an increase in the magnetic memory of the saline solution, and therefore a better emissive power when it exists.

The treatment by nascent oxygen can be performed using electrodes linked to a determined potential, at least one of which is immersed in the compensation medium. The treatment by nascent oxygen of the compensation medium 4 can be performed at at least one of the following stages:
before the exposure to the inverted radiation,
during the exposure to the radiation, in which case the receiving device 12 will be adapted to receive the electrode-based means needed,
after the exposure to the inverted radiation, for example upstream or downstream of a possible succussion step.

The nascent oxygen is also used as a stability tracer.

The treatment by the ELF low-frequency channel makes it possible in particular to take account of the frequencies associated with switching in a communicating appliance, for example when it uses TDMA access techniques.

The refinement provided by the embodiment consists in the use of a new technique for treating the compensation medium, providing in particular a greater control of the parameters involved in the processing of the signal.

The compensation oscillator formed by the assembly 1 that has just been described has been the subject of biological studies relating to the embryonic mortality of chickens' eggs exposed to different electromagnetic polluters. It shows the significant improvement of the electromagnetic biocompatibility of the polluter thanks to the addition of the appropriate compensation oscillator. As an indication, the results observed with a laptop computer as the polluting device can be summarized by:
control group: 16% embryonic mortality,
group exposed without protection: 61% embryonic mortality,
group exposed protected by compensation oscillator 1: 31% embryonic mortality.

It will be apparent to those skilled in the art that the invention lends itself to numerous variants, both in its hardware implementation and its applications, and the parameters and adjustments, which will be adapted to the conditions of use. It will be noted that the compensation medium can be any substance, liquid, gel or solid, organized in a molecular microcrystalline network, which oscillates either naturally (quartz, for example), or after physical-chemical treatment, or mainly electromagnetically.

1. Method of preparing a compensation medium intended for protection against the biological effects of the electromagnetic radiations emitted by an appliance, wherein it comprises the steps of:
analyzing a first signal corresponding to electromagnetic radiations emitted by the appliance,
producing a second signal which differs from the first signal by an inversion relating to at least one signal parameter, and
applying the second signal, in electromagnetic radiation form, to the compensation medium.

2. Method according to claim 1, wherein said inversion relates to at least one of the following signal parameters:
the phase,
the polarity of the return wave,
at least a part of the spectrum.

3. Method according to claim 1 wherein the inversion is produced by a digital processing of said signal.

4. Method according to claim 1, wherein the inversion comprises at least one out of:
a Fourier transform,
a Merlin-Fourier transform,
a Laplace transform, in particular for the relatively high frequencies.

5. Method according to claim 1, wherein the second signal is produced by transforming the first signal by means of an electronic processing device.

6. Method according to claim 1, wherein the second signal is produced by synthesis.

7. Method according to claim 6, wherein the second signal is produced from parameter control data for a reference generator operating in the frequency range for which protection is required.

8. Method according to claim 1, wherein it comprises the steps of:
extracting the first signal from the electromagnetic emission from a source appliance roughly of the same type as the appliance for which the protection is intended, the source appliance and the compensation medium being placed respectively in a radiation detecting device and in a radiation emitting device that are mutually matched, the emitting device supplying the signal to the compensation medium from the electromagnetic radiation of the source device,
subjecting the first signal to the inversion upstream of the emitting device, transforming it into said second signal,
supplying said second as input to the emitting device such that the latter produces in response an electromagnetic radiation with corresponding inversion, received by the compensation medium.

9. Method according to claim 1, wherein the relatively high and relatively low frequency bands of the first signal are processed separately.

10. Method according to claim 1, wherein the radiation in a relatively high frequency band of the second signal is transmitted to the compensation medium by means of a multi-dipole antenna, and the radiation in a relatively low frequency band of the second signal is transmitted to the compensation medium by means of a winding.

11. Method according to claim 1, wherein the first signal is also subjected to a filtering for the purpose of eliminating the high-order harmonics.

12. Method according to claim 1, wherein the compensation medium is also subjected to a treatment by a nascent oxidant, for example nascent oxygen.

13. Method according to claim 12, wherein it also comprises a step for defining the stability of the compensation medium, by analyzing the nascent oxygen in the latter.

14. Method of protection against the biological effects of the non-ionizing electromagnetic radiations emitted by an appliance, wherein it comprises the steps of:
preparing a compensation medium according to claim 1, where said first signal corresponds to the electromagnetic radiation emitted by the appliance,
packaging a quantity of the duly prepared compensation medium in a support, and
physically connecting said support to said appliance.

15. Device specifically provided for the preparation of a compensation medium intended for protection against the biological effects of the non-ionizing electromagnetic radiations emitted by an appliance, wherein it comprises:
means for analyzing a first signal corresponding to electromagnetic radiations emitted by the appliance,
means for producing a second signal which differs from the first signal by an inversion relating to at least one signal parameter, and
means for applying the second signal, in electromagnetic radiation form, to the compensation medium.

16. Device according to claim 15, wherein said inversion relates to at least one out of the following signal parameters:
the phase,
the polarity of the return wave,
at least a part of the spectrum.

17. Device according to claim 15, wherein it comprises digital signal processing means intended to perform said inversion.

18. Device according to claim 15, wherein the inversion comprises at least one out of:
a Fourier transform,
a Merlin-Fourier transform,
a Laplace transform, in particular for the relatively high frequencies.

19. Device according to claim 15, wherein it comprises electronic signal processing means for producing the second signal by transforming the first signal.

20. Device according to claim 15, wherein it comprises synthesis means for producing the second signal.

21. Device according to claim 20, wherein it comprises means of establishing parameter control data for controlling a reference generator operating in the frequency range for which protection is required in order to produce the second signal.

22. Device according to claim 15, wherein it comprises at least two separate channels respectively intended for the processing of the first signal at relatively high and relatively low frequency bands.

23. Device according to claim 15, wherein it comprises a multi-dipole antenna for transmitting to the compensation medium the radiation in a relatively high frequency band of the second signal, and a winding for transmitting to the compensation medium the radiation in a relatively low frequency band of the first signal.

24. Device according to claim 15, wherein it also comprises filtering means in order to eliminate the high-order harmonics.

25. Device according to wherein it also comprises means for submitting the compensation medium to a treatment by nascent oxygen.

26. Support intended to be physically connected to an appliance emitting non-ionizing electromagnetic radiations, wherein it contains a quantity of compensation medium treated according to claim 1.

27. Appliance emitting non-ionizing electromagnetic radiations, wherein it incorporates the support according to claim 26.

28. Method according to claim 2 wherein the inversion is produced by a digital processing of said signal.

29. Device according to claim 16 wherein it comprises digital signal processing means intended to perform said inversion.
